(19) United States
(12) Patent Application Publication
(10) Pub. No.: US 2025/0065065 A1
(43) Pub. Date: Feb. 27, 2025

(54) FLOW GENERATOR CHASSIS ASSEMBLY WITH SUSPENSION SEAL

(71) Applicant: ResMed Pty Ltd, Bella Vista (AU)

(72) Inventors: Richard Llewelyn JONES, Sydney (AU); Saad NASR, Sydney (AU); Joern LANGE, Sydney (AU); Ernie Wei-Chih TSAI, Sydney (AU); Barton John KENYON, Sydney (AU); Geoffrey CRUMBLIN, Sydney (AU)

(21) Appl. No.: 18/939,737

(22) Filed: Nov. 7, 2024

Related U.S. Application Data
(63) Continuation of application No. 17/408,654, filed on Aug. 23, 2021, now Pat. No. 12,161,800.
Application No. 17/408,654 is a continuation of application No. 15/434,358, filed on Feb. 16, 2017, now Pat. No. 11,129,948.
Application No. 15/434,358 is a continuation of application No. 14/556,343, filed on Dec. 1, 2014, now Pat. No. 9,610,416.
Application No. 14/556,343 is a continuation of application No. 12/794,206, filed on Jun. 4, 2010, now Pat. No. 8,931,481.
(60) Provisional application No. 61/184,124, filed on Jun. 4, 2009.

(51) Int. Cl. A61M 16/00; A61M 16/10; F04D 29/42; F04D 29/60

(52) U.S. Cl. CPC A61M 16/0066; A61M 16/0003; A61M 16/105; F04D 29/4226; F04D 29/601; A61M 2016/0027; A61M 2016/003; A61M 2016/0033; A61M 16/0069; A61M 16/107; A61M 2205/3334; A61M 2205/3368; A61M 2205/42; A61M 2205/75; Y10T 29/49826

(57) ABSTRACT

A chassis assembly of a flow generator that includes a lower chassis portion, an upper chassis portion, and a seal sandwiched between the lower chassis portion. The lower chassis portion and the upper chassis portion together form a blower receptacle configured to hold a blower. The lower chassis portion and the upper chassis portion together form an inlet chamber comprising a gas inlet of the chassis assembly and configured to convey the breathable gas to the blower receptacle. The lower chassis portion and the upper chassis portion together form an outlet chamber comprising a gas outlet of the chassis assembly and configured to convey the pressurized breathable gas from the blower receptacle to the gas outlet of the chassis assembly. The seal is configured so that the breathable gas enters the chassis assembly on the first side of the seal and exits the chassis assembly on the second side of the seal.

### CROSS REFERENCE TO RELATED APPLICATIONS

This application is a continuation of U.S. application Ser. No. 17/408,654, filed Aug. 23, 2021, now allowed, which is a continuation of U.S. application Ser. No. 15/434,358, filed Feb. 16, 2017, now U.S. Pat. No. 11,129,948, which is a continuation of U.S. application Ser. No. 14/556,343, filed Dec. 1, 2014, now U.S. Pat. No. 9,610,416, which is a continuation of U.S. application Ser. No. 12/794,206, filed Jun. 4, 2010, now U.S. Pat. No. 8,931,481, which claims the benefit of U.S. Application No. 61/184,124, filed Jun. 4, 2009, each of which is incorporated herein by reference in its entirety.

### FIELD OF THE INVENTION

The present technology relates to a flow generator for generating a flow of breathable gas to be delivered to a patient. In an embodiment, the flow generator may be used in a positive airway pressure (PAP) device used for the delivery of respiratory therapy to a patient. Examples of such therapies are Continuous Positive Airway Pressure (CPAP) treatment, Non-Invasive Positive Pressure Ventilation (NIPPV), Variable Positive Airway Pressure (VPAP), and Bi-Level Positive Airway Pressure. The therapy is used for treatment of various respiratory conditions including Sleep Disordered Breathing (SDB) and more particularly Obstructive Sleep Apnea (OSA). The present technology also relates to a seal for the flow generator. The present technology further relates to methods of assembling a flow generator.

### BACKGROUND

Flow generators are used to generate a flow of breathable gas for treatment of various respiratory conditions. The flow generator may be configured to be connected to a patient interface, for example a mask, to deliver the flow of breathable gas to the patient's airways. The flow generator may also be configured to be connected to a humidifier configured to store a supply of water for humidifying the flow of breathable gas prior to delivery to the patient.

A blower is provided in the flow generator to generate the flow of breathable gas. A blower may comprise a motor comprising a rotating part including, for example, a shaft having an impeller(s) mounted thereon, and a non-rotating part. The operation of the motor may generate noise and vibration. As the therapy may be delivered to the patient during sleep, the generation of noise and vibration may reduce the effectiveness of the therapy.

The flow generator may also include electronics configured to control the operation of the motor of the blower to vary the pressure of the flow of breathable gas that is generated by the blower. The electronics may be damaged by the flow of breathable gas generated by the blower, for example by debris drawn into the flow generator.

A flow sensor may be provided to measure the volume of the flow of breathable gas generated by the blower. Gaps in the flow generator chassis assembly may allow portions of the flow of breathable gas to bypass the flow sensor, thus providing an inaccurate measure of the flow.

### BRIEF SUMMARY

According to one aspect, a flow generator chassis assembly is provided with a seal that is configured to mount the blower.

According to another aspect, the seal is configured to align the motor of the blower.

According to still another aspect, the seal is configured as a suspension for the blower.

According to a further aspect, the seal is configured to seal between high and low pressure sides of the flow generator chassis assembly.

According to yet another aspect, the seal is configured to seal an inlet air flow path and/or an outlet air flow path of the flow generator chassis assembly.

According to another aspect, the seal is configured to seal a portion of the flow generator chassis assembly from which a wire of the blower motor extends out of the chassis assembly.

According to a further aspect, the seal is configured to seal between acoustic chambers of the flow generator chassis assembly.

According to still another aspect, the seal is configured to seal an air flow path through the flow generator chassis assembly from electronics contained in the flow generator chassis assembly.

According to yet another aspect, the seal may be configured to include a flow sensor tube seal and a manifold connection to the flow sensor.

According to one sample embodiment of the technology, a seal is provided for a chassis assembly of a flow generator configured to provide a flow of breathable gas. The chassis assembly includes an upper chassis and a lower chassis. The seal comprises a blower receptacle sealing portion configured to seal a blower receptacle of the chassis assembly configured to receive a blower configured to generate the flow of breathable gas; a flow sensor scaling portion configured to seal a flow sensor provided in the chassis assembly and configured to measure the flow of breathable gas; a blower mount configured to receive a portion of the blower and mount the blower to the seal; and a blower mount suspension configured to connect the blower mount to the blower receptacle sealing portion, wherein in an assembled condition the seal and the chassis assembly define a flow path from an inlet of the chassis assembly to the blower receptacle of the chassis assembly to an outlet of the chassis assembly.

According to another sample embodiment, a flow generator for generating a flow of breathable gas comprises a chassis assembly comprising a lower chassis and an upper chassis, the lower chassis and the upper chassis defining a blower receptacle; a blower provided in the blower receptacle; and a seal according to at least the preceding paragraph.

According to still another sample embodiment, a method of assembling a flow generator, for example as described above, comprises mounting the blower to the blower mount of the seal; positioning the seal on the lower chassis; routing the wire of the blower through the grommet of the seal; and fastening the upper chassis and the lower chassis together.

According to yet another sample embodiment, a method of assembling a flow generator, for example as described above, comprises mounting the blower to the blower mount of the seal; aligning the seal on the upper chassis; positioning the lower chassis on the seal; and fastening the upper chassis and the lower chassis together.

According to a further sample embodiment, a flow generator for generating a pressurized flow of breathable gas comprises a chassis comprising a lower chassis and an upper chassis, the lower chassis and the upper chassis defining an enclosure; a blower provided in the enclosure and configured to generate a flow of breathable gas from air drawn through an inlet in the chassis and deliver the flow of breathable gas to an outlet in the chassis; and a blower mount between the lower chassis and the upper chassis that mounts the blower in the enclosure. The blower mount comprises a blower receptacle that receives a lower portion of the blower, a blower mount outlet configured to deliver the flow of breathable gas from the blower to the outlet in the chassis, and a seal in engagement with the upper chassis to provide a sealed air flow path from an inlet of a upper chassis blower receptacle to the blower mount outlet.

Other aspects, features, and advantages of the technology will become apparent from the following detailed description when taken in conjunction with the accompanying drawings, which are a part of this disclosure and which illustrate, by way of example, principles of the technology.

### BRIEF DESCRIPTION OF THE DRAWINGS

The accompanying drawings facilitate an understanding of the various embodiments of this invention. In such drawings:

FIG. 1 is a top perspective view of a flow generator according to a sample embodiment;

FIG. 2 is a bottom perspective of the flow generator of FIG. 1;

FIG. 3 is a bottom perspective of the flow generator of FIG. 1;

FIGS. 4-7 are perspective views of the upper chassis of the flow generator according to a sample embodiment;

FIG. 8 is a top perspective view of a flow generator seal according to a sample embodiment;

FIG. 9 is a bottom perspective view of the flow generator seal of FIG. 8;

FIG. 10 is a perspective view of a lower chassis of the flow generator according to a sample embodiment;

FIG. 11 is a perspective view of the lower chassis and the flow generator seal prior to assembly;

FIG. 12 is a perspective view of the lower chassis and flow generator seal after assembly;

FIG. 13 is an exploded perspective view of the flow generator seal and a blower of the flow generator according to a sample embodiment;

FIG. 14 is a perspective view of the blower and flow generator seal of FIG. 13 in an assembled condition;

FIG. 15 is a perspective view of the flow generator seal and blower in an assembled condition;

FIG. 16 is a perspective of the flow generator assembly prior to assembly according to one sample embodiment;

FIG. 17 is a perspective view of the flow generator seal and the upper chassis according to another sample embodiment of a method of assembly;

FIG. 18 is a perspective view of the flow generator prior to fastening of the upper chassis, flow generator seal, and lower chassis; and

FIG. 19 is a perspective view of the flow generator including an outer casing.

### DETAILED DESCRIPTION OF THE ILLUSTRATED EMBODIMENTS

The following description is provided in relation to several embodiments which may share common characteristics and features. It is to be understood that one or more features of any one embodiment may be combinable with one or more features of the other embodiments. In addition, any single feature or combination of features in any of the embodiments may constitute additional embodiments.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The term “air” will be taken to include breathable gases, for example air with supplemental oxygen. It is also acknowledged that the blowers described herein may be designed to pump fluids other than air.

### Chassis

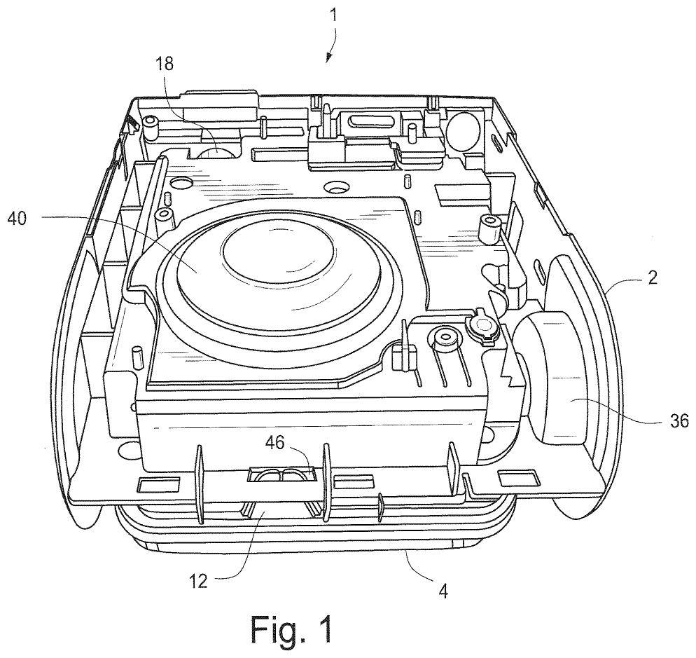

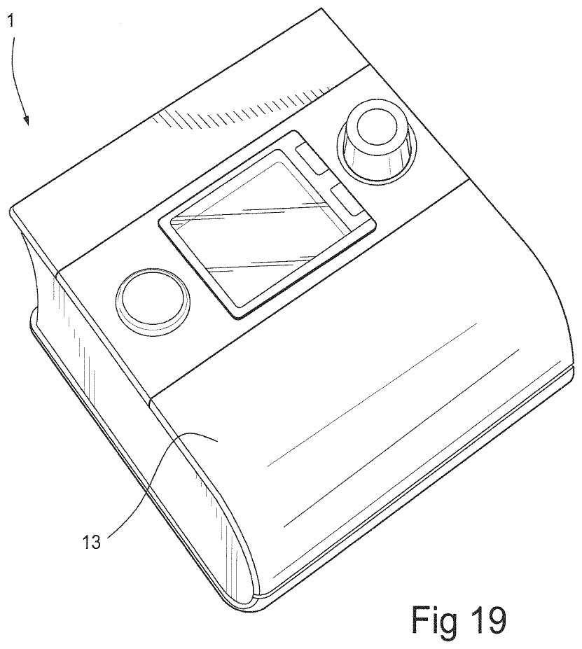

Referring to FIG. 1, a chassis assembly of a flow generator 1 includes an upper chassis 2 and a lower chassis 4. The upper chassis 2 and the lower chassis 4 may be formed of, for example, plastic. The upper chassis 2 includes an air flow outlet 36 that is configured to be connected to a tube or conduit for delivery of a flow of breathable gas to a patient interface that is configured to engage the patient to deliver the flow of breathable gas. It should also be appreciated that the air flow outlet 36 is configured to be connected to the inlet of a humidifier configured to store a supply of water for humidifying the flow of breathable gas. Referring to FIG. 19, the flow generator 1 may comprise an outer casing 13 that covers and/or surrounds the chassis assembly.

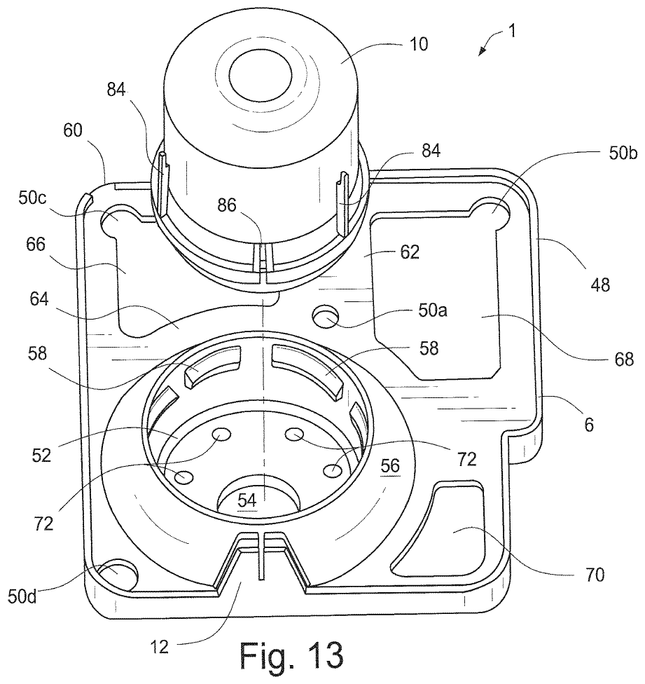

The upper chassis 2 also includes an upper chassis receptacle top portion 40 that is configured to receive the top of a blower 10 (FIG. 13) described in more detail below. The blower is configured to generate the flow of breathable gas, e.g. in the range of about 2-30 cm H₂O, from air drawn through an air inlet 18 provided in the upper chassis 2.

A grommet 12 of a flow generator seal extends through a grommet slot 46 provided in the upper chassis 2.

### Blower Mount

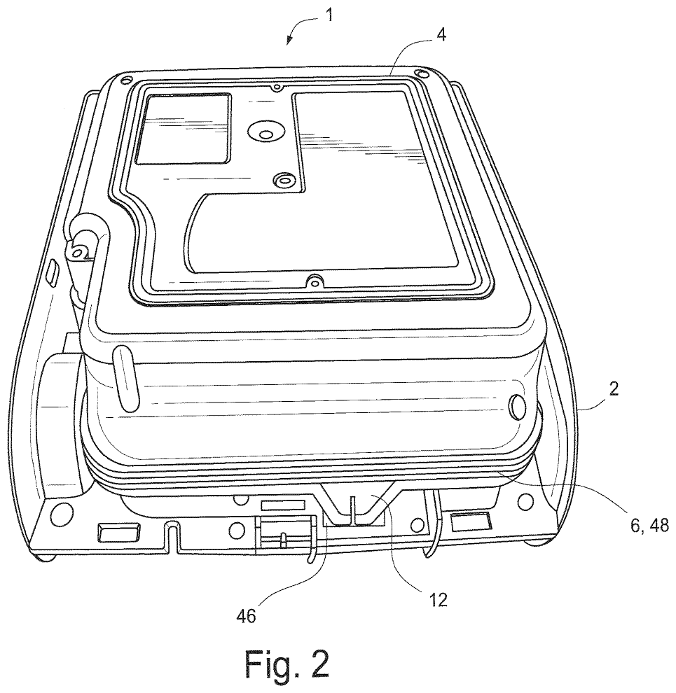

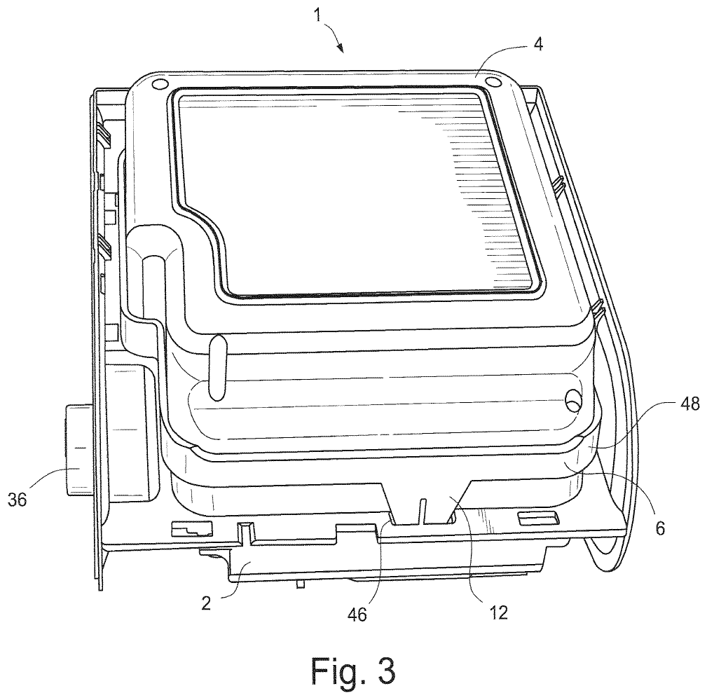

Referring to FIGS. 2, 3, 8 and 9, the blower mount, or flow generator seal, 6 is provided between the upper chassis 2 and the lower chassis 4. As discussed in more detail below, the flow generator seal 6 provides a sealed inlet air path for the flow generator 1. The sealed inlet air path directs the inlet air flow through an inlet filter and prevents the air flow from leaking through gaps in the case formed by the upper chassis 2 and the lower chassis 4. The inlet air flow thus does not flow over the electronics. The sealing of the air flow also reduces noise leakage through the gaps in the case. It should be appreciated, however, that the flow generator seal 6 may not include an inlet air path seal.

As shown in FIGS. 2, 3, 8, 9, 12-15 and 17, the flow generator seal 6 includes a flow generator seal skirt 48 having a T-shaped cross section that extends around the perimeter of the flow generator seal 6 and engages the upper chassis 2 and the lower chassis 4 to seal the junction between the upper chassis 2 and the lower chassis 4.

The seal 6 may be formed of a flexible material, for example silicone. The seal may also be formed so as to be elastically deformable, for example to stretch, to permit the seal to engage various portions of the flow generator chassis assembly and/or blower while in a deformed, e.g. stretched, condition. The seal may also be transparent or translucent. In other sample embodiments, the seal may also comprise a frame or support, e.g. a rigid support skeleton, that may be, for example, overmolded onto the flexible material (or the flexible material of the seal may be overmolded onto the frame or support), that provides additional locations for other components of the flow generator and/or aids in assembly of the flow generator.

### Upper Chassis

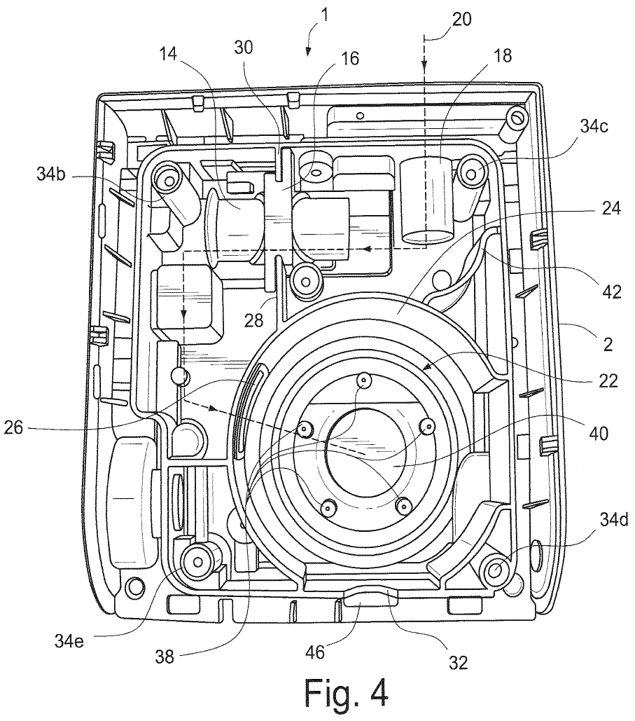

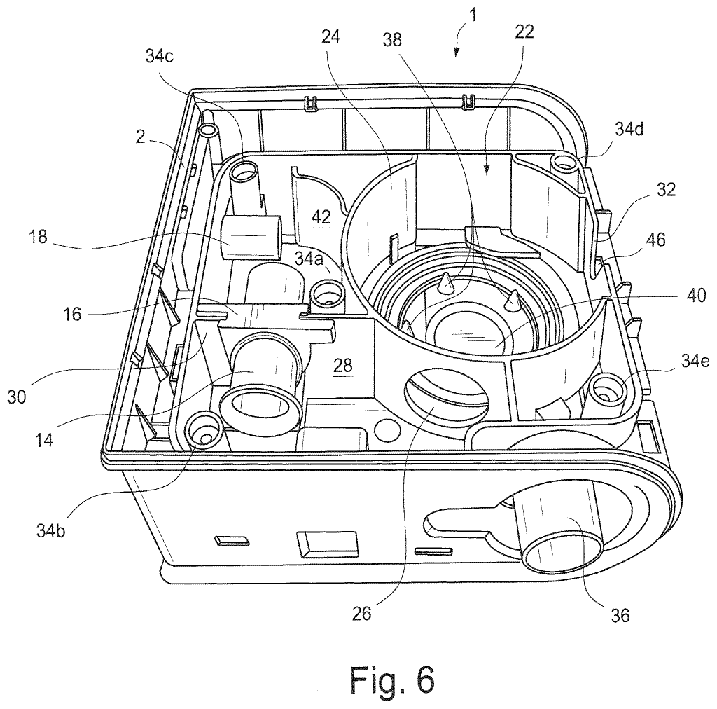

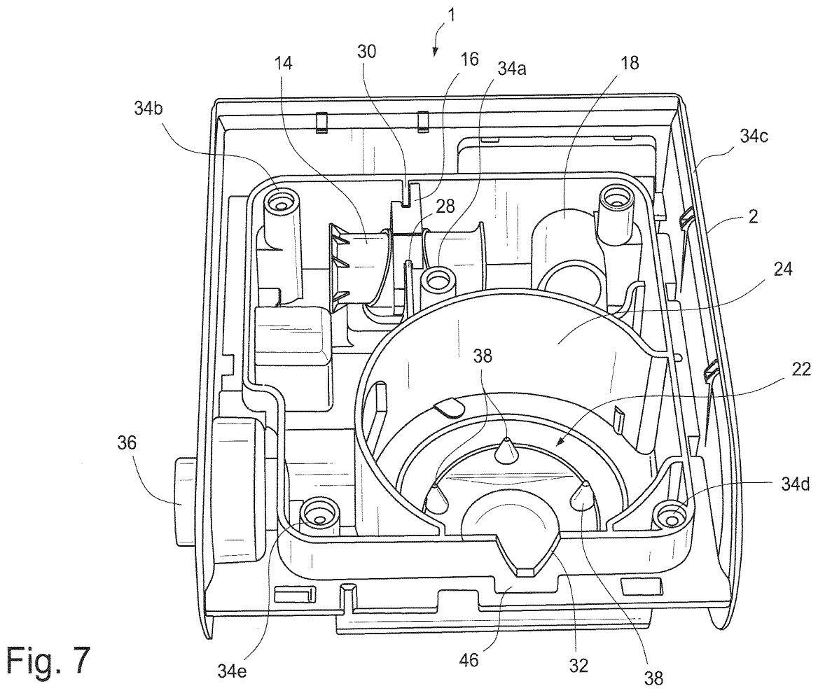

Referring to FIGS. 4-7, the upper chassis 2 comprises the air inlet 18. An air inlet flow path 20 is established by the operation of a blower that is provided in an upper chassis blower receptacle 22 when the upper chassis 2, the flow generator seal 6, and the lower chassis 4 are assembled. The air inlet flow path 20 includes a flow sensor 14 that is supported in the upper chassis 2 between a pair of interior walls 28, 30. A flow sensor seal 16 is provided to prevent the inlet air flow path 20 from bypassing the flow sensor 14. It should be appreciated that the flow sensor seal 16 may be provided separately from the seal 6. It should also be appreciated that the flow sensor seal 16 may be integrally formed with the seal 6 and include the manifold connection to the flow sensor 14. It should be further appreciated that other sensors, e.g. pressure and/or thermistor, may be sealed, for example by integrally forming other sensor seals with the flow generator seal 6.

After passing through the flow sensor 14, the inlet air flow path 20 passes through a side wall air inlet 26 provided in the upper chassis blower receptacle side wall 24. The upper chassis blower receptacle 22 comprises a plurality of upper chassis bump stops 38 and a grommet notch 32 that is configured to receive the grommet 12 of the flow generator seal 6 that is configured to hold the electrical wire extending from the blower that is contained in the upper chassis blower receptacle 22. The upper chassis 2 also comprises a grommet slot 46 that the grommet 12 passes through.

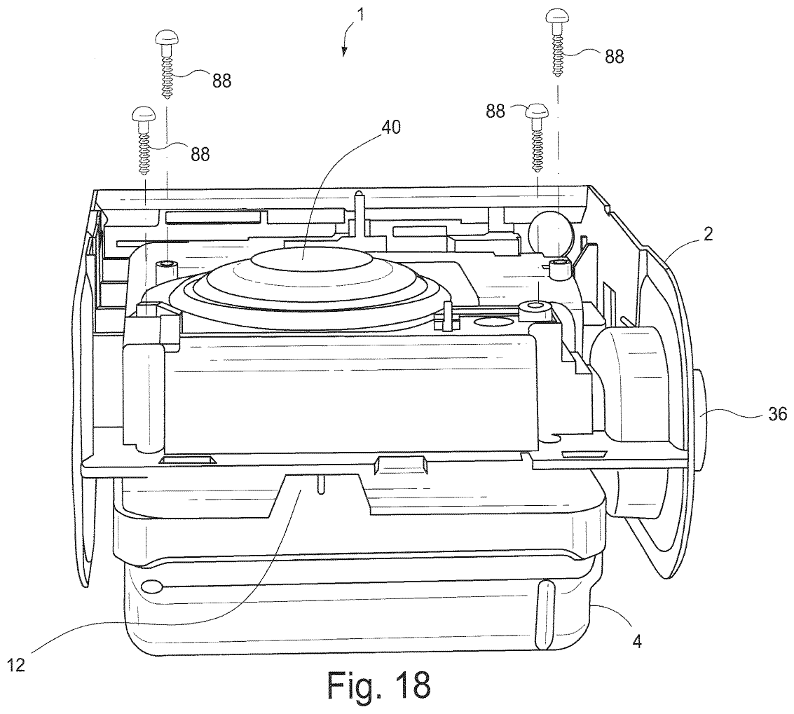

A plurality of upper chassis fastener receptacles 34a-34e are provided in the upper chassis 2 and are configured to receive fasteners 88 (FIG. 18) that are configured to assemble the upper chassis 2, the flow generator seal 6 and the lower chassis 4 in to the flow generator 1.

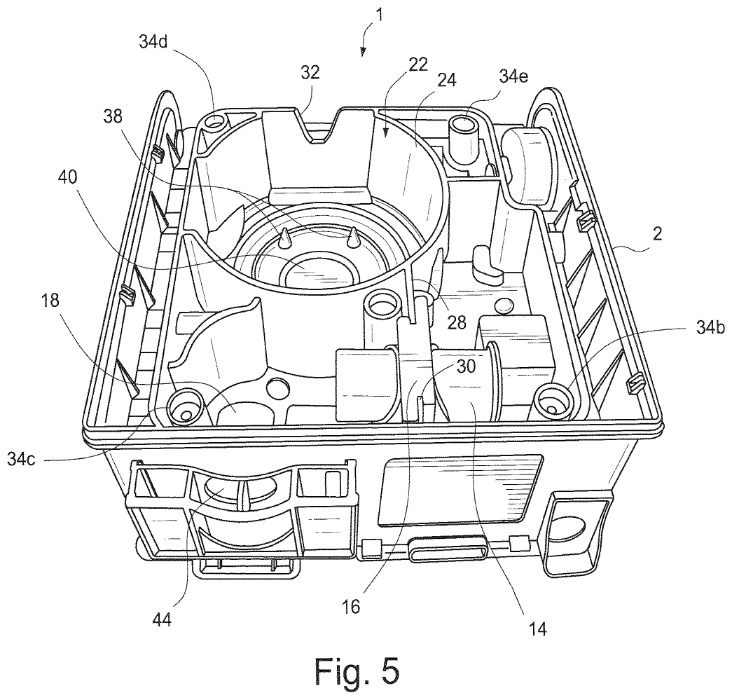

As shown in FIG. 5, the air inlet 18 of the upper chassis 2 includes an air inlet aperture 44 formed on a side wall of the upper chassis 2. It should be appreciated that an air inlet filter may be provided at the air inlet aperture 44 to filter air drawn in to the flow generator 1 by the blower.

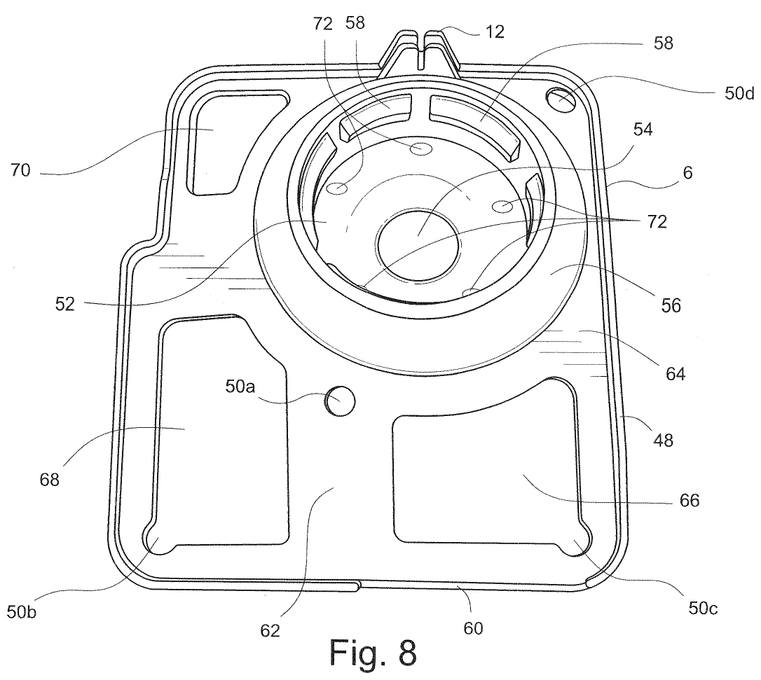

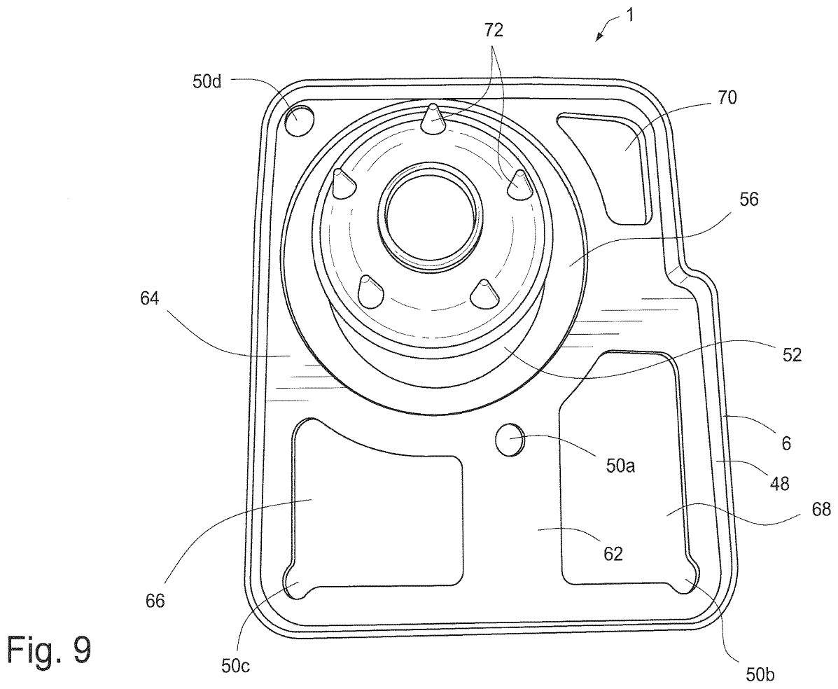

Referring to FIGS. 8 and 9, the flow generator seal 6 comprises the T-shaped skirt 48 that extends around the perimeter of the flow generator seal 6. As shown in FIG. 8, a flow generator seal inlet portion 60 is provided on the flow generator seal 6. The T-shaped skirt 48 does not extend above the perimeter of the flow generator seal 6 in this region of the flow generator seal inlet portion 60 which is configured to be aligned with the air inlet aperture 44 and the air inlet 18 of the upper chassis 2. The skirt is L-shaped in cross section in the region of the seal inlet portion 60 and extends below the perimeter of the flow generator seal 6 and engages the perimeter of the lower chassis 4.

On the side opposite of the flow generator seal inlet portion 60, the flow generator seal 6 comprises the wire grommet 12 that is configured to hold the wire extending from the motor of the blower. A flow sensor seal portion 62 extends between the side including the inlet portion 60 and a blower receptacle and mount suspension 56 provided on the flow generator seal 6. The wire grommet 12 may be located at other positions on the seal 6, however locating the wire grommet 12 adjacent to the blower receptable and mount suspension 56 minimizes the length of the wires.

The blower receptacle and mount suspension 56 has a frusto-conical shape and supports a blower receptacle and mount 52 that is configured to receive a portion of the blower. As shown in FIG. 9, the blower receptacle and mount 52 comprises a plurality of flow generator seal bump stops 72 that are configured to prevent the blower bumping into the lower chassis 4.

The blower receptacle and mount 52 also comprises a plurality of blower receptacle and mount ribs 58 spaced around a periphery of the receptacle and mount 52 that are configured to engage the blower. The blower receptacle mount 52 also comprises a blower receptacle and mount outlet 54 through which the flow of breathable gas generated by the blower passes through to the outlet 36 of the flow generator 1.

The flow generator seal 6 includes a plurality of flow generator seal fastener openings 50a-50d that correspond to the upper chassis fastener receptacles 34a-34d. The fastener openings 50a-50d allow the fasteners to pass through to permit the upper chassis 2, the flow generator seal 6, and a lower chassis 4 to be assembled in to the flow generator 1.

A blower receptacle seal portion 64 extends circumferentially around the blower receptacle and mount suspension 56 and is configured to provide a seal around the blower received in the blower receptacle and mount 52. An air inlet seal opening 66 is provided adjacent to the blower receptacle seal portion 64 and the air inlet flow path 20 passes the air inlet seal opening in to the side wall air inlet 26 of the side wall 24 of the blower receptacle 22 of the upper chassis 2.

The air inlet seal opening 66 is provided adjacent to the blower receptacle seal portion 64 in the region of the air inlet 18 of the upper chassis 2 to permit the inlet air flow path to enter the flow generator 1 through the air inlet 18. An air flow outlet seal opening 70 is provided adjacent to the blower receptacle seal portion 64 so that the flow of breathable gas produced by the blower can pass to the outlet 36 of the flow generator 1.

### Lower Chassis

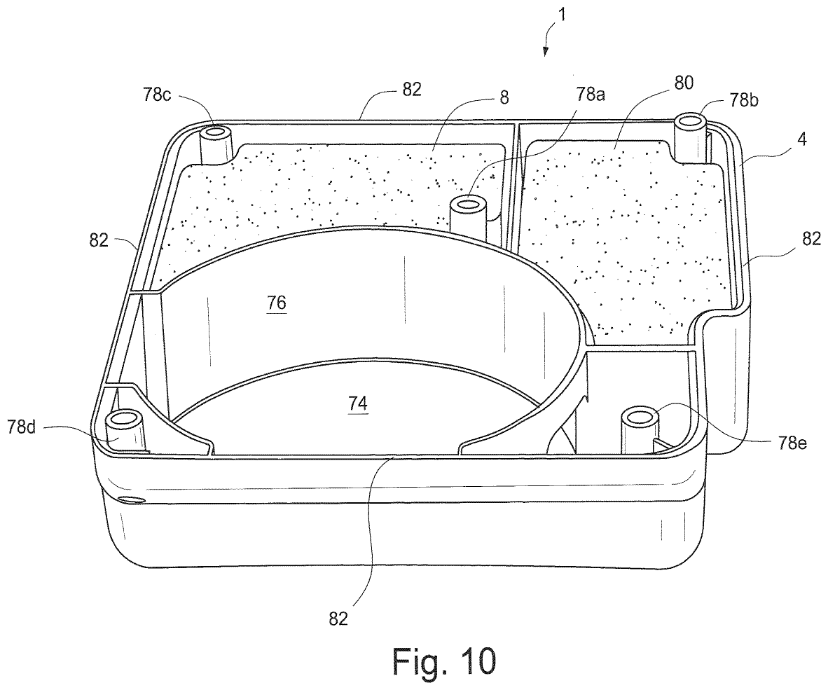

Referring to FIG. 10, the lower chassis 4 includes a lower chassis blower receptacle 74 that comprises a lower chassis blower receptacle side wall 76 that is configured to receive a portion of the blower. A plurality of lower chassis fastener receptacles 78a-78e are provided that correspond to the upper chassis fastener receptacle openings 34a-34e and the flow generator seal fastener openings 50a-50d.

Referring still to FIG. 10, the compartment of the lower chassis 4 that corresponds to the blower inlet seal opening 68 of the flow generator seal 6 may comprise a foam 80. The compartment of the lower chassis 4 that corresponds to the air inlet seal opening 66 of the flow generator seal 6 may comprise a foam 8.

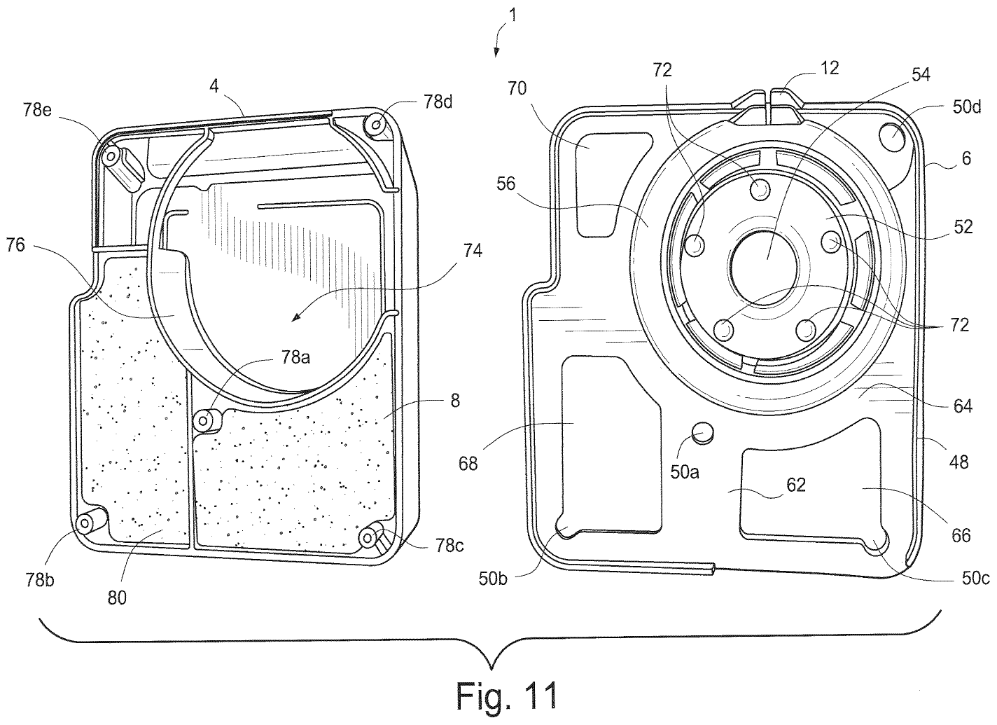

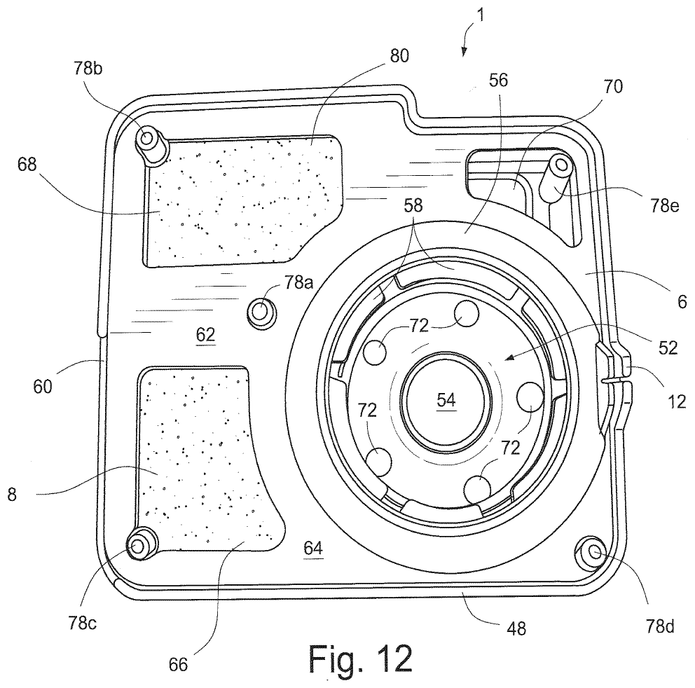

Referring to FIG. 11, the flow generator seal 6 has a perimeter 82 that corresponds in shape to the perimeter of the lower chassis 4. As shown in FIG. 12, in the assembled condition, the flow generator seal 6 is provided around the lower chassis 4 so that the T-shaped skirt 48 of the flow generator seal 6 sealingly engages the perimeter of the lower chassis 4. The lower chassis fastener receptacles 78a-78d extend through the flow generator seal openings 50a-50d. The foam 8 is exposed through the air inlet seal opening 66 and the foam 80 is exposed through the blower inlet seal opening 68.

### Blower and Blower Mount

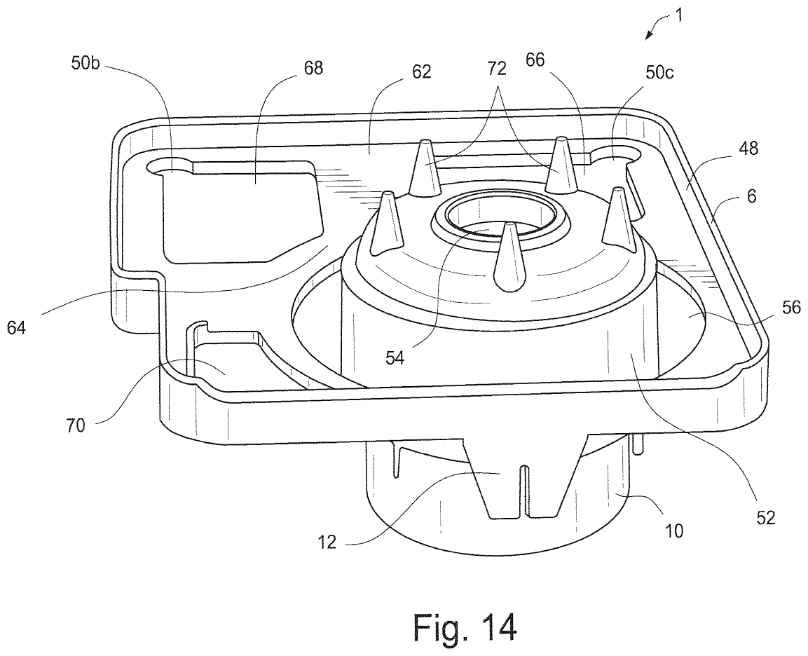

Referring to FIGS. 13 and 14, the blower 10 is configured to be received in the blower receptacle mount 52 of the flow generator seal 6. The blower 10 may be as disclosed in, for example, in WO 2007/048205 A1, WO 2007/048206 A1, WO 2007/134405 A1, or U.S. Patent Application Publication 2008/0304986 A1, the entire contents of each being incorporated herein by reference. The motor and blower may be configured to provide a flow of breathable gas at a constant pressure, a variable pressure, or a bi-level pressure.

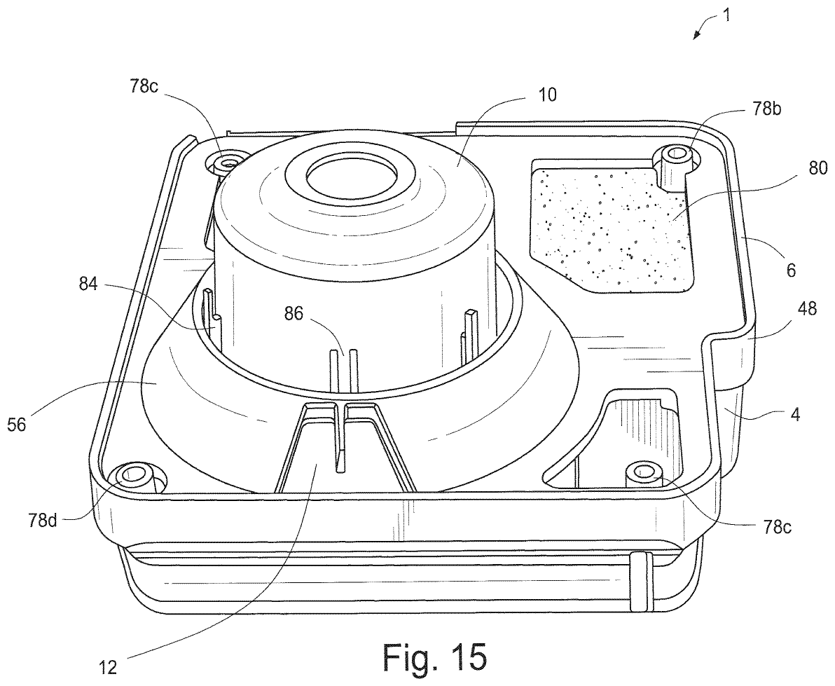

The outer periphery of the blower 10 may comprise blower alignment tabs 84 that are configured to be received between adjacent blower receptacle and mount ribs 58. The blower 10 also comprises a blower wire guide 86 that guides the wire from the motor of the blower 10 which is configured to be aligned with the wire grommet 12 of the flow generator seal 6, as shown in FIG. 15.

In use, when the blower 10 is running, the upper chassis 2 is the low pressure side and the lower chassis 4 is the high pressure side of the seal 6. The pressurized chamber causes the blower 10 to lift off the lower chassis 4 such that the blower 10 is suspended by the seal 6. The flow generator seal bump stops 72 and the upper chassis bump stops 38 are configured to prevent the blower 10 from bumping into the lower chassis 4 and the upper chassis 2, respectively.

### Flow Generator Assembly

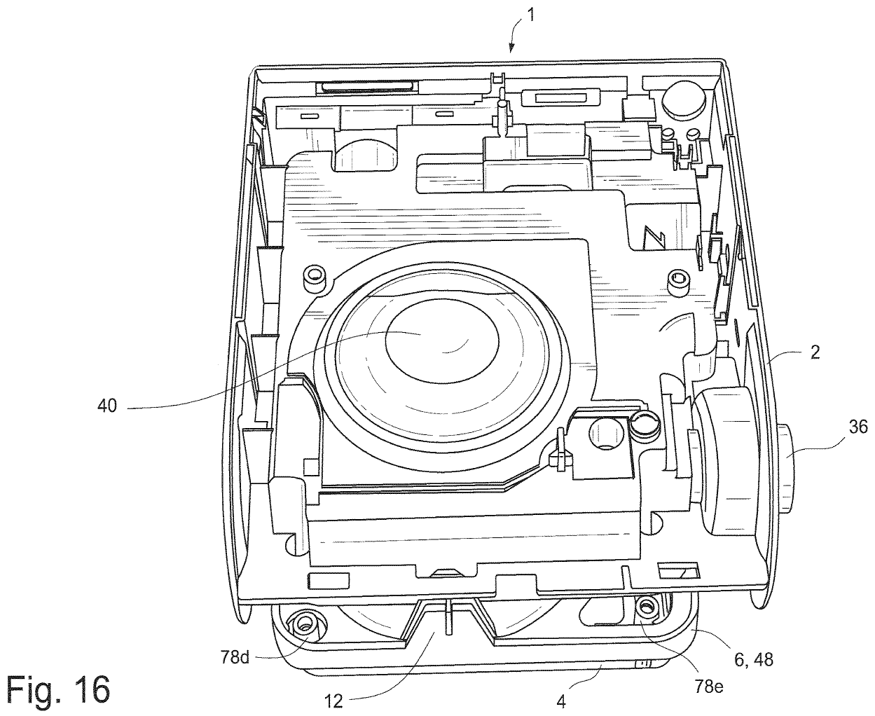

As shown in FIGS. 13-16, according to one sample embodiment of an assembly method, the flow generator seal 6 may be assembled with the blower 10, as shown in FIGS. 13 and 14. The flow generator seal and blower assembly 6, 10 may then be assembled on to the lower chassis 4 as shown in FIG. 15. Then, as shown in FIG. 16, the upper chassis 2 may be assembled to the lower chassis 4 and the flow generator seal and blower assembly 6, 10 shown in FIG. 15. The upper chassis 2 is assembled to the lower chassis 4 so that the blower 10 is received in the upper chassis receptacle top portion 40.

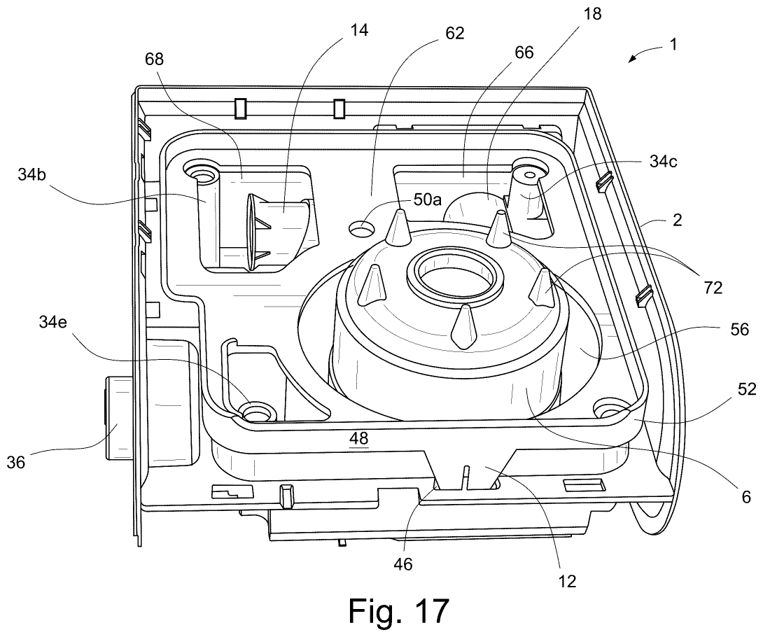

According to another sample embodiment of the assembly of the flow generator 1, the flow generator seal 6 may be assembled to the upper chassis 2 as shown in FIG. 17. The lower chassis 4 may then be assembled to the upper chassis 2 and the assembly fastened together by fasteners 88, as shown in FIG. 18.

According to another sample assembly embodiment, the upper chassis 2 is assembled with the flow tube 14. The blower 10 is then assembled with the flow generator seal 6. The wires of the motor of the blower 10 are routed through the blower wire guide 86 and the wire grommet 12 of the flow generator seal 6. The assembled flow generator seal and blower assembly 6, 10 is then aligned with the upper chassis 2 so that the wire grommet 12 aligns with the wire grommet notch 32 of the upper chassis 2. The assembled flow generator seal and blower assembly 6, 10 is then positioned on the upper chassis 2 and the flow generator seal 6 is stretched to make sure that the T-shaped skirt 48 is positioned outside the interior walls of the upper chassis 2.

The lower chassis 4 is then assembled with the foams 8, 80 and the lower chassis 4 is positioned on the flow generator seal 6. The T-shaped skirt 48 is then stretched around the perimeter 82 of the lower chassis 4 so that it fully surrounds the lower chassis 4. In an alternative embodiment, the seal may be sized to be a clearance fit over the perimeter 82 of the lower chassis 4, which may make assembly easier. The assembly is then fastened together by inserting the fasteners 88 in to the corresponding fastener receptacles 34a-34d, 78a-78d, respectively, of the upper chassis 2 and the lower chassis 4 and through the flow generator seal fastener openings 50a-50d. A fifth fastener may then be inserted into the fastener openings 34e, 78e of the upper chassis 2 and the lower chassis 4, respectively. The fifth fastener would pass through the air flow outlet seal opening 70 of the seal 6.

According to still another sample embodiment of the assembly method, the lower chassis 4 is assembled with the foams 8, 80. The blower 10 is then assembled with the flow generator seal 6 and positioned on the lower chassis 4. The T-shaped skirt 48 may be stretched so that the flow generator seal is sitting around the perimeter 82 of the lower chassis and the fastener openings 50a-50d of the flow generator seal 6 are provided around the fastener openings 78a-78d of the lower chassis 4. The wire of the motor of the blower 10 is routed through the blower wire guide 86 of the blower 10 and through the wire grommet 12 of the flow generator seal 6.

The upper chassis 2 is then assembled by securing the flow sensor 14 in to the upper chassis 2 with the flow sensor seal 16. The upper chassis 2 is then aligned with the lower chassis 4 so that the wire grommet 12 passes through the wire grommet slot 46 of the upper chassis 2 and is received in to the wire grommet notch 32 of the upper chassis 2. The assembly of the flow generator 1 is then completed by securing the fasteners 88 in to the fastener receptacles 34a-34d, 78a-78d, respectively, of the upper chassis 2 and the lower chassis 4 passing through the fastener openings 50a-50d in the flow generator seal 6. A fifth fastener may then be inserted into the fastener openings 34e, 78e of the upper chassis 2 and the lower chassis 4, respectively. The fifth fastener would pass through the air flow outlet seal opening 70 of the seal 6.

The seal mounts the motor and/or blower, provides alignment for the motor, and/or provides a suspension for the motor and/or blower. The seal also provides a seal between the high and low pressure sides of the motor and/or blower. The seal may seal the inlet air flow path and/or the outlet air flow path. The wire grommet seal seals the portion of the flow generator chassis assembly from which the wire of the motor extends. The seal also seals between acoustic chambers of the flow generator chassis assembly and/or seals the air flow path from the electronics of the flow generator. The seal may also include a flow sensor tube seal and manifold connection to the flow sensor. The provision of a seal capable of performing any or all of these functions reduces the part count and assembly time of the flow generator. The air flow path, not including the flow sensor, of the flow generator may be formed of three parts, i.e. the upper and lower chassis and the seal. The use of the upper and lower chassis along with the seal to form the air flow path allows the refinement of the upper and lower chassis, which may be formed for example of plastic, to achieve a high level of manufacturability at a lower cost.

While the technology has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment. Furthermore, each individual component of any given assembly, one or more portions of an individual component of any given assembly, and various combinations of components from one or more embodiments may include one or more ornamental design features. In addition, while the invention has particular application to patients who suffer from OSA, it is to be appreciated that patients who suffer from other illnesses (e.g., congestive heart failure, diabetes, morbid obesity, stroke, bariatric surgery, etc.) can derive benefit from the above teachings. Moreover, the above teachings have applicability with patients and non-patients alike in non-medical applications.

1. (canceled)

2. A chassis assembly of a flow generator configured to provide a flow of breathable gas at a continuously positive pressure with respect to ambient air pressure to an entrance of a patient's airways including at least an entrance of a patient's nares, wherein the flow generator is configured to maintain a therapy pressure in a range of about 2 cm H₂O to about 30 cm H₂O above ambient air pressure throughout the patient's respiratory cycle, while the patient is sleeping, to ameliorate sleep disordered breathing, the chassis assembly comprising:
a lower chassis portion;
an upper chassis portion; and
a seal sandwiched between the lower chassis portion and the upper chassis portion,
wherein the lower chassis portion and the upper chassis portion together form a blower receptacle configured to hold a blower;
wherein the lower chassis portion and the upper chassis portion together form an inlet chamber comprising a gas inlet of the chassis assembly and configured to convey the breathable gas to the blower receptacle,
wherein the lower chassis portion and the upper chassis portion together form an outlet chamber comprising a gas outlet of the chassis assembly and configured to convey the pressurized breathable gas from the blower receptacle to the gas outlet of the chassis assembly,
wherein the seal seals a perimeter of the blower receptacle, seals a perimeter of the inlet chamber, and seals a perimeter of the outlet chamber, and
wherein the seal is configured so that the breathable gas enters the chassis assembly on the first side of the seal and the pressurized breathable gas exits the chassis assembly on the second side of the seal.

3. The chassis assembly of claim 2, wherein the seal comprises:
a blower sealing portion that seals the perimeter of the blower receptacle;
an inlet sealing portion that is adjacent to the blower receptacle sealing portion and seals the perimeter of the inlet chamber; and
an outlet sealing portion that is adjacent to the blower receptacle sealing portion and seals the perimeter of the outlet chamber.

4. The chassis assembly of claim 2, further comprising:
a T-shaped skirt extending around a portion of the seal and configured to seal a junction between the upper chassis and the lower chassis.

5. The chassis assembly of claim 4, wherein the T-shaped skirt does not extend around the seal in a region of the gas inlet of the chassis assembly, and the seal comprises an L-shaped skirt in the region of the inlet of the chassis assembly.

6. The chassis assembly of claim 2, wherein the seal comprises a plurality of openings configured to receive a respective plurality of fasteners configured to fasten the upper chassis and the lower chassis together.

7. The chassis assembly of claim 2, wherein the seal comprises a grommet configured to receive a wire of the blower.

8. The chassis assembly of claim 2, wherein an interior of the inlet chamber is filled with foam.

9. The chassis assembly of to claim 8, wherein foam is positioned within at least a portion of the outlet chamber.

11. The chassis assembly of claim 2, wherein the blower receptacle comprises an outlet corresponding to an outlet of the blower.

12. The chassis assembly of claim 2, wherein the seal is formed of a flexible material.

13. The chassis assembly of claim 2, wherein the seal is formed of an elastically deformable material.

14. The chassis assembly of claim 2, wherein the seal is formed of silicone.

15. The chassis assembly of claim 2, wherein the seal is transparent or translucent.

16. The chassis assembly of claim 2, wherein the seal is a continuous body.

17. The chassis assembly of claim 2, wherein the seal comprises a plurality of openings that allow communication through the seal between the upper chassis portion and the lower chassis portion.

18. The chassis assembly of claim 2, wherein the seal comprises an opening that allows the pressurized breathable gas in the outlet chamber to flow between the upper chassis portion and the lower chassis portion.

19. A flow generator configured to provide a flow of breathable gas at a continuously positive pressure with respect to ambient air pressure to an entrance of a patient's airways including at least an entrance of a patient's nares, wherein the flow generator is configured to maintain a therapy pressure in a range of about 2 cm H₂O to about 30 cm H₂O above ambient air pressure throughout the patient's respiratory cycle, while the patient is sleeping, to ameliorate sleep disordered breathing, the flow generator comprising:
the chassis assembly of claim 1; and
a blower positioned within the chassis assembly.

20. The flow generator of claim 19, further comprising:
an air inlet fluidly connected to the gas inlet of the chassis assembly; and
an air delivery tube connector fluidly connected to gas outlet of the chassis assembly and configured to connect to an air delivery tube.

21. A respiratory system comprising:
the flow generator of claim 20; and
an air delivery tube connectable to the air delivery tube connector.
